(19) United States
(12) Patent Application Publication
(10) Pub. No.: US 2008/0194492 A1
(43) Pub. Date: Aug. 14, 2008

(54) Plant Extracts for the Treatment of Incresed Bone Resorption

(76) Inventors: Rudolf Brenneisen, Bern (CH); Roman Conrad Muhlbauer, Rapperswil (CH); Herbert Wetli, Basel (CH)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/580,186

(22) PCT Filed: Nov. 25, 2004
(86) PCT No.: PCT/EP2004/013413

§ 371 Date: Sep. 21, 2007

Related U.S. Application Data
(60) Provisional application No. 60/624,446, filed on Nov. 2, 2004.

(30) Foreign Application Priority Data
Nov. 26, 2003 (GB) 0327527.8

(51) Int. Cl. A61K 38/05; A61K 31/197; A61P 3/02; A61K 38/16; A61K 33/06

(52) U.S. Cl. 514/19; 514/563; 424/682; 514/12

(57) ABSTRACT

The present invention concerns the use of γ-glutamyl-peptide, for example γ-L-glutamyltrans-S-1-propenyl-L-cysteine sulfoxide, in the treatment or prevention of diseases or conditions which are characterized by increased bone resorption, such as Paget's disease, tumor-induced bone disease or osteoporosis, inhibits dose-dependently the resorption activity of osteoclasts, the minimal effective dose being about 2 mM.

### TECHNICAL FIELD

The present invention relates to plant derived material with bone resorption inhibiting activity.

### BACKGROUND OF THE INVENTION

The most common metabolic bone disorder is osteoporosis. Osteoporosis can be generally defined as the reduction in the quantity of bone, either from the reduction in bone formation or the acceleration of bone resorption, in either event the result is a decrease in the amount of skeletal tissue. Bone mass in adult humans decreases with age, leading to an increased risk of fractures. Osteoporotic fractures, besides causing suffering to the patient, are a major burden to health care, as the direct expenditure for osteoporosis and associated fractures is around $17 billion/year in the U.S.A. alone (Melton, L. J.; Heaney, R. P. Too much medicine? Or too little? Bone 2003, 32, 327-331).

Osteoclasts (bone resorbing cells) are responsible for the excavation of a portion of bone during the resorption process. After resorption, osteoblasts (bone forming cells) appear, which then refill the resorbed portion with new bone.

In young healthy adults, the rate at which the osteoclasts and osteoblasts are formed and operate maintains a balance between bone resorption and bone formation. However, as a normal consequence of aging, an imbalance in this remodeling process develops, resulting in loss of bone. As imbalance continues over time, the reduction in bone mass and thus bone strength leads to fractures.

Many compositions and methods are described in the medical literature for the treatment of osteoporosis. For example, estrogens, calcitonin and bisphosphonates are known to be effective inhibitors of bone resorption.

A nutritional approach would be an inexpensive means to achieve this goal. However, the effects of the nutritional strategies recommended today are rather modest. Indeed, even the effect of calcium in milk on the relative risk of hip fractures seems to be restricted to the 10% of the female population with the lowest intake of calcium (Kanis, J. A. The use of calcium in the management of osteoporosis. Bone 1999, 24, 279-290). Thus, research into novel nutritional strategies preventing bone loss is needed.

EP 980250 discloses, inter alia, nutritional, e.g. veterinary, or pharmaceutical compositions, e.g. animal medicines, comprising a plant extract or concentrate of allium and their use for the treatment or prophylaxis of a disease or condition which is characterized by increased bone resorption, such as Paget's disease, tumor-induced bone disease or particularly osteoporosis. The subject matter of EP 980250 is herein incorporated by reference to this application.

The addition of 7% of onion to the diet of rats decreases bone resorption and increases bone mineral content in growing rats (Mühlbauer, R. C.; Li, F. Effect of vegetables on bone metabolism. Nature 1999, 401, 343-344). This effect is independent of the base excess of onion (Mühlbauer, R. C.; Lozano, A.; Reinli, A. Onion and a mixture of vegetables, salads and herbs affect bone resorption in the rat by a mechanism independent of their base excess. J Bone Miner Res 2002, 17, 1230-1236).

Furthermore, an ethanolic extract from onion prevented bone loss in an osteoporosis model and inhibited the resorption activity of osteoclasts in vitro (Ingold, P.; Kneissel, M.; Mühlbauer, R. C.; Gasser, J. A. Extracts from onion prevent tibial cortical and cancellous bone loss induced by a high phosphate/low protein diet in aged retired breeder rats. Bone 1998, 23, S387, Abstract #W388; and Mühlbauer, R. C.; Li, F.; Guenther, H. L. Common vegetables consumed by humans potently modulate bone metabolism in vitro and in vivo. Bone 1998, 23, S387, Abstract #W391). This suggested that the inhibitory activity of onion on bone resorption could be due to a pharmacologically active compound.

A first fractionation of the ethanolic extract showed no activity in vivo of the flavonoid containing fraction, but instead the activity eluted with the more polar compounds. This rendered the easy approach of testing the flavonoids abundant in onion obsolete. As the polar material also inhibited the resorption activity of osteoclasts, this in vitro culture system could be used as bioassay, prompting us to undertake the isolation and identification of the unknown compound(s) in onion inhibiting bone resorption.

However, the active constituents of allium responsible for the bone resorption inhibitory effect have not yet been described.

### SUMMARY OF THE INVENTION

The active constituent of allium responsible for the bone resorption inhibiting effect, may be found in an hydrophilic, ethanolic extract of allium such as Allium cepa. The active constituent having a potent inhibitory effect on bone resorption was identified as a γ-glutamyl-peptide, for example a γ-glutamyl-alkyl-cysteine sulfoxide or γ-glutamyl-alkenyl-cysteine sulfoxide, further example a γ-L-glutamyl-trans-S-1-propenyl-L-cysteine sulfoxide.

As used herein, the term allium refers to the genus allium (latin for garlic, a member of the onion family) and includes for example any member of the botanical species Allium cepa (onion), Allium ascalonicum (shallot), Allium ampeloprasum (leek/great-headed-garlic), Allium porrum (leek), Allium schoenoprasum (chive), Allium ursinum (bear's garlic), Allium sativum (garlic) or Allium fistulosum (bunching onion). Preferred species are Allium ascalonicum (shallot), Allium porrum (leek), Allium cepa (onion) and Allium ursinum (bear's garlic, also known as bear paw garlic), particularly the latter two, whereby Allium cepa is particularly preferred.

The active ingredient of the invention may be isolated from allium, e.g. Allium cepa, e.g. from the whole eatable part of the vegetable, by fractionation, e.g. in vitro bioassay guided fractionation, for example as described hereinbelow Alternatively, the active ingredient of the invention, in particular the cis-form thereof, may be obtained by full or semi chemical synthesis, for example as readily known to one skilled in the art

γ-glutamyl-peptide, for example a γ-glutamyl-alkyl-cysteine sulfoxide or γ-glutamyl-alkenyl-cysteine sulfoxide, further example a γ-L-glutamyl-trans-S-1-propenyl-L-cysteine sulfoxide, are hereinafter referred to as active ingredient of the invention. According to the invention, the active ingredient of the invention can be in a concentrate form.

Accordingly, in one aspect the present invention relates to the use of -glutamyl-peptide, e.g. in a concentrate form, for example a γ-glutamyl-alkyl-cysteine sulfoxide or γ-glutamyl-alkenyl-cysteine sulfoxide, further example γ-L-glutamyl-trans-S-1-propenyl-L-cysteine sulfoxide, e.g. in a concentrate form, in the preparation of a medicament or nutritional formulation, e.g. animal medicine or veterinary composition, for the treatment, testing for, or prophylaxis of a disease or condition which is characterized by increased bone resorption, such as Paget's disease, tumor-induced bone disease or osteoporosis. In a further aspect, the invention relates to nutritional, e.g. veterinary, or pharmaceutical compositions, e.g. animal medicine, comprising the active ingredient of the invention, e.g. in a concentrate form and optionally:
(a) an acceptable carrier;
(b) a calcium source;
(c) at least one energy source selected from the group consisting of carbohydrate, fat and nitrogen sources;
(d) Vitamin D; or any combination thereof.

In one aspect of the invention, the γ-glutamyl-peptide of a medicament or nutritional formulation, inhibits dose-dependently the resorption activity of osteoclasts

In another embodiment of the invention the medicament or nutritional formulation, has a dose of γ-glutamyl-peptide of at least 2 mM.

The invention further provides a method for the treatment, testing for, or prophylaxis of a disease or condition which is characterized by increased bone resorption, such as Paget's disease, tumor-induced bone disease or osteoporosis, comprising the administration of a medicament or nutritional formulation to a human or an animal, e.g. a mammal, said medicament or nutritional formulation comprising the active ingredient of the invention, e.g. in a concentrate form, in an amount which is effective for inhibiting bone resorption.

In yet a further aspect, the present invention provides for a method of inhibiting bone resorption which method comprises administering to a human or an animal, e.g. a mammal, in need thereof an effective amount of a composition comprising the active ingredient of the invention, e.g. in a concentrate form.

In yet a further aspect the present invention provides for the use of comprising the active ingredient of the invention, e.g. in a concentrate form, in the dietary management of increased bone resorption.

In one aspect, the invention provides the active ingredient of the invention, e.g. γ-L-glutamyl-trans-S-1propenyl-L-cysteine sulfoxide, e.g. in a concentrate form, obtained by fractionation of an hydrophilic, ethanolic extract of Allium cepa, which fractionation comprises
(a) obtaining an hydrophilic, ethanolic extract of Allium cepa, hereinafter referred to as fraction A, by using adsorption column chromatography, e.g. Amberlite® XAD-4,
(b) separating saccharides from fraction A by using reversed-phase medium pressure liquid chromatography (RP-MPLC) to obtain fraction A1,
(c) further separating saccharides from fraction A1 by using normal-phase medium pressure liquid chromatography (NP-MPLC), e.g. using a mobile phase chosen from
   (c1) methylethylketone-acetic acid-methanol, e.g. in a ratio of 6:5:3 (v/v),
   (c2) acetone-water-hydrochloric acid 37%, e.g. in a ratio of 9 ml:1 ml:1 drop,
   (c3) n-butanole-acetic acid-diethylether-water, e.g. in a ratio of 9:6:3:1 (v/v),
   (c4) chloroform-methanol-water, e.g. in a ratio of 6.4:5:1,
      for example using chloroform-methanol-water 6.4:5:1 as mobile phase, to obtain fraction A1-4,
(d) further fractionation by semi-preparative reversed-phase HPLC (SP-RP-HPLC), e.g. using as solvent an isocratic water/acetonitrile system buffered with e.g. 0.00625% formic acid to obtain fraction A1-4C.

In a further aspect, the invention provides the active ingredient of the invention, e.g. γ-L-glutamyl-trans-S-1-propenyl-L-cysteine sulfoxide, e.g. in a concentrate form, obtained by in vitro bioassay guided fractionation of an hydrophilic, ethanolic extract of Allium cepa, which bioassay guided fractionation comprises
(a) obtaining an hydrophilic, ethanolic extract of Allium cepa, hereinafter referred to as fraction A, by using adsorption column chromatography, e.g. Amberlite® XAD-4,
(b) assessing in vitro the bone resorbing inhibitory activity by using the osteoclast pit assay,
(c) separating saccharides from fraction A by using reversed-phase medium pressure liquid chromatography (RP-MPLC) to obtain fraction A1,
(d) assessing in vitro the bone resorbing inhibitory activity by using the osteoclast pit assay,
(e) further separating saccharides from fraction A1 by using NP-MPLC, e.g. using a mobile phase chosen from
   (e1) methylethylketone-acetic acid-methanol, e.g. in a ratio of 6:5:3 (v/v),
   (e2) acetone-water-hydrochloric acid 37%, e.g. in a ratio of 9 ml:1 ml:1 drop,
   (e3) n-butanole-acetic acid-diethylether-water, e.g. in a ratio of 9:6:3:1 (v/v),
   (e4) chloroform-methanol -water, e.g. in a ratio of 6.4:5:1,
      for example using chloroform-methanol-water 6.4:5:1 as mobile phase, to obtain fraction A1-4,
(d) assessing in vitro the bone resorbing inhibitory activity by using the osteoclast pit assay,
(e) further fractionation by semi-preparative reversed-phase HPLC (SP-RP-HPLC), e.g. using as solvent an isocratic water/acetonitrile system buffered with 0.00625% formic acid to obtain fraction A1-4C,
(f) assessing in vitro the bone resorbing inhibitory activity by using the osteoclast pit assay.

The compound of fraction A1-4C may be analyzed by mass spectroscopy, e.g. using a HPLC-ESI-MS equipment, e.g. using an MS equipped with a quadruple ion trap (QIT). Fragmentation may be achieved by colliding the positively charged, ionized molecule with helium gas, e.g. using a collision energy of 35%.

The structure of the compound of fraction A1-4C may be further confirmed by ESI-MS-MS after acid hydrolysis.

Furthermore, the structure of the compound of fraction A1-4C may be confirmed by nuclear magnetic resonance (NMR) spectroscopy, e.g. ¹H-NNMR, ¹J_{CH}-COSY NMR, ¹H-H-COSY, and/or ⁿJ_{CH}-COSY NMR, e.g. using D₂O as solvent and trimethylsilyl-propansulfonic acid as external standard, or other techniques for the analysis of the compound of fraction A1-4C are readily known to one skilled in the art.

### BRIEF DESCRIPTION OF THE DRAWINGS

FIG. 1. Is a graph illustrating the effect on bone resorption in rats fed a purified diet containing either 1 g of dried onion (open square), or 639 mg of dried alcoholic onion extract corresponding to 1 g onion, (open triangle), or 595 mg of the dried hydrophilic fraction A corresponding to 1 g onion (closed triangle), or finally 7.1 mg of the dried lipophilic fraction B corresponding to 1 g of onion (open diamond). The 95% confidence interval (CI; 1.96×SEM) of the untreated control group is given as box with dotted background. Values are means±SEM. n=6 for control and 5 for treated. Mean values outside the 95% CI are significantly different from control (p<0.05) ((Previously published in Mühlbauer, R. C.; Lozano, A.; Reinli, A.; Wetli, H. Various selected vegetables, fruits, mushrooms and red wine residue inhibit bone resorption in rats. J Nutr 2003, 133, 3592-3597).

FIG. 2. Is a graph illustrating the dose-dependent effect of the hydrophilic fraction A and of calcitonin on in vitro resorption activity of osteoclasts. Fraction A was added to the medium at the concentration of 1, 10 and 30 mg/mL, and calcitonin at 10⁻¹² M. Data presentation: the values of the treated groups (n=8) are given as the ratio treated/untreated±SEM. The 95% CI (1.96×SEM) of the untreated group (n=16) is given as mean±SEM (box with dotted background). Mean values outside the 95% CI of the untreated group are significantly different (p<0.05) ( Previously published in J Nutr 2003, 133, 3592-3597).

FIG. 3. Is a graph illustrating the effect of various onion fractions and of calcitonin on in vitro resorption activity of osteoclasts. Fractions A 1-4, A 1-4B and A 1-4C were added to the medium at concentrations of 2.28, 0.43 and 0.53 mg/mL respectively. Calcitonin was used at the dose of 10⁻¹² M. Data presentation as described in FIG. 2.

FIG. 4. Is a graph illustrating the effect of GPCS and of calcitonin on in vitro resorption activity of osteoclasts. GPCS was added to the medium at concentrations of 2, 4, and 8 mM. Calcitonin was used at the dose of 10⁻¹¹ M. Two separate experiments were performed: one without addition of parathyroid hormone (panel A) and one in which to all cultures PTH (10⁻⁸ M) was added to stimulate bone resorption. Data presentation as described in FIG. 2.

FIG. 5. Is a structural depiction of a γ-L-glutamyl-trans-S-1-propenyl-L-cysteine-sulfoxide (GPCS)

### DETAILED DESCRIPTION OF THE INVENTION

After long and exhaustive testing it has now surprisingly been found that the active constituent of allium responsible for the bone resorption inhibiting effect, may be found in an hydrophilic, ethanolic extract of allium such as Allium cepa. The active constituent having a potent inhibitory effect on bone resorption was identified as a γ-glutamyl-peptide, for example a γ-glutamyl-alkyl-cysteine sulfoxide or γ-glutamyl-alkenyl-cysteine sulfoxide, further example a γ-L-glutamyl-trans-S-1-propenyl-L-cysteine sulfoxide.

γ-glutamyl-peptide, for example a γ-glutamyl-alkyl-cysteine sulfoxide or γ-glutamyl-alkenyl-cysteine sulfoxide, further example a γ-L-glutamyl-trans-S-1-propenyl-L-cysteine sulfoxide, are hereinafter referred to as active ingredient of the invention. According to the invention, the active ingredient of the invention can be in a concentrate form.

Accordingly, in one aspect the present invention relates to the use of γ-glutamyl-peptide, e.g. in a concentrate form, for example a γ-glutamyl-alkyl-cysteine sulfoxide or γ-glutamyl-alkenyl-cysteine sulfoxide, further example γ-L-glutamyl-trans-S-1-propenyl-L-cysteine sulfoxide, e.g. in a concentrate form, in the preparation of a medicament or nutritional formulation, e.g. animal medicine or veterinary composition, for the treatment, testing for, or prophylaxis of a disease or condition which is characterized by increased bone resorption, such as Paget's disease, tumor-induced bone disease or osteoporosis. In a further aspect, the invention relates to nutritional, e.g. veterinary, or pharmaceutical compositions, e.g. animal medicine, comprising the active ingredient of the invention, e.g. in a concentrate form.

The invention further provides a method for the treatment, testing for, or prophylaxis of a disease or condition which is characterized by increased bone resorption, such as Paget's disease, tumor-induced bone disease or osteoporosis, comprising the administration of a medicament or nutritional formulation to a human or an animal, e.g. a mammal, said medicament or nutritional formulation comprising the active ingredient of the invention, e.g. in a concentrate form, in an amount which is effective for inhibiting bone resorption.

In yet a further aspect, the present invention provides for a method of inhibiting bone resorption which method comprises administering to a human or an animal, e.g. a mammal, in need thereof an effective amount of a composition comprising the active ingredient of the invention, e.g. in a concentrate form.

In yet a further aspect the present invention provides for the use of comprising the active ingredient of the invention, e.g. in a concentrate form, in the dietary management of increased bone resorption.

Osteoporosis as used herein includes osteoporosis induced by hormone deficiency (e.g. postmenopausal) and old age, as well as secondary osteoporosis such as osteoporosis secondary to steroid treatment or secondary to malnutrition caused by anorexia nervosa.

The active ingredient of the invention may be isolated from allium, e.g. Allium cepa, e.g. from the whole eatable part of the vegetable, by fractionation, e.g. in vitro bioassay guided fractionation, for example as described hereinbelow Alternatively, the active ingredient of the invention, in particular the cis-form thereof, may be obtained by full or semi chemical synthesis, for example as readily known to one skilled in the art

In one aspect, the invention provides the active ingredient of the invention, e.g. γ-L-glutamyl-trans-S-1propenyl-L-cysteine sulfoxide, e.g. in a concentrate form, obtained by fractionation of an hydrophilic, ethanolic extract of Allium cepa, which fractionation comprises
(a) obtaining an hydrophilic, ethanolic extract of Allium cepa, hereinafter referred to as fraction A, by using adsorption column chromatography, e.g. Amberlite® XAD-4,
(b) separating saccharides from fraction A by using reversed-phase medium pressure liquid chromatography (RP-MPLC) to obtain fraction A1,
(c) further separating saccharides from fraction A1 by using normal-phase medium pressure liquid chromatography (NP-MPLC), e.g. using a mobile phase chosen from
   (c1) methylethylketone-acetic acid-methanol, e.g. in a ratio of 6:5:3 (v/v),
   (c2) acetone-water-hydrochloric acid 37%, e.g. in a ratio of 9 ml:1 ml:1 drop,
   (c3) n-butanole-acetic acid-diethylether-water, e.g. in a ratio of 9:6:3:1 (v/v),
   (c4) chloroform-methanol-water, e.g. in a ratio of 6.4:5:1,
      for example using chloroform-methanol-water 6.4:5:1 as mobile phase, to obtain fraction A1-4,
(d) further fractionation by semi-preparative reversed-phase HPLC (SP-RP-HPLC), e.g. using as solvent an isocratic water/acetonitrile system buffered with e.g. 0.00625% formic acid to obtain fraction A1-4C.

In a further aspect, the invention provides the active ingredient of the invention, e.g. γ-L-glutamyl-trans-S-1propenyl-L-cysteine sulfoxide, e.g. in a concentrate form, obtained by in vitro bioassay guided fractionation of an hydrophilic, ethanolic extract of Allium cepa, which bioassay guided fractionation comprises
(a) obtaining an hydrophilic, ethanolic extract of Allium cepa, hereinafter referred to as fraction A, by using adsorption column chromatography, e.g. Amberlite® XAD-4,
(b) assessing in vitro the bone resorbing inhibitory activity by using the osteoclast pit assay,
(c) separating saccharides from fraction A by using reversed-phase medium pressure liquid chromatography (RP-MPLC) to obtain fraction A1,
(d) assessing in vitro the bone resorbing inhibitory activity by using the osteoclast pit assay,
(e) further separating saccharides from fraction A1 by using NP-MPLC, e.g. using a mobile phase chosen from
   (e1) methylethylketone-acetic acid-methanol, e.g. in a ratio of 6:5:3 (v/v),
   (e2) acetone-water-hydrochloric acid 37%, e.g. in a ratio of 9 ml:1 ml:1 drop,
   (e3) n-butanole-acetic acid-diethylether-water, e.g. in a ratio of 9:6:3:1 (v/v),
   (e4) chloroform-methanol-water, e.g. in a ratio of 6.4:5:1,
      for example using chloroform-methanol-water 6.4:5:1 as mobile phase, to obtain fraction A1-4,
(d) assessing in vitro the bone resorbing inhibitory activity by using the osteoclast pit assay,
(e) further fractionation by semi-preparative reversed-phase HPLC (SP-RP-HPLC), e.g. using as solvent an isocratic water/acetonitrile system buffered with 0.00625% formic acid to obtain fraction A1-4C,
(f) assessing in vitro the bone resorbing inhibitory activity by using the osteoclast pit assay.

It will be appreciated that the osteoclast pit assay is a well established in vitro model of bone resorption and readily known to one skilled in the art. Briefly, medium containing a sample to be tested, e.g. 30 mg or less of freeze-dried hydrophilic fraction A per ml, is added to osteoclasts of new-born rats settled on ivory slices. After 24 hours of incubation, the tartrate-resistant acid phosphatase positive multi-nucleated cells, i.e. osteoclasts, are counted. Subsequently, the number of resorption pits is determined. Activity is calculated as the ratio of resorption pits per osteoclast and compared to a negative control, e.g. medium containing 10% fetal bovine serum and to a positive control, e.g. 10⁻¹² M calcitonin. For the analysis of statistical significance, the ratios of the treated groups±their respective SEMs are compared to the 95% confidence interval of the SEM of the negative control.

The compound of fraction A1-4C may be analyzed by mass spectroscopy, e.g. using a HPLC-ESI-MS equipment, e.g. using an MS equipped with a quadruple ion trap (QIT). Fragmentation may be achieved by colliding the positively charged, ionized molecule with helium gas, e.g. using a collision energy of 35%.

The structure of the compound of fraction A1-4C may be further confirmed by ESI-MS-MS after acid hydrolysis.

Furthermore, the structure of the compound of fraction A1-4C may be confirmed by nuclear magnetic resonance (NMR) spectroscopy, e.g. ¹H-NNMR, ¹J_{CH}-COSY NMR, ¹H-H-COSY, and/or ⁿJ_{CH}-COSY NMR, e.g. using D₂O as solvent and trimethylsilyl-propansulfonic acid as external standard.

It will be appreciated that such techniques for the analysis of the compound of fraction A1-4C are readily known to one skilled in the art.

As used herein, the term allium refers to the genus allium (latin for garlic, a member of the onion family) and includes for example any member of the botanical species Allium cepa (onion), Allium ascalonicum (shallot), Allium ampeloprasum (leek/great-headed-garlic), Allium porrum (leek), Allium schoenoprasum (chive), Allium ursinum (bear's garlic), Allium sativum (garlic) or Allium fistulosum (bunching onion). Preferred species are Allium ascalonicum (shallot), Allium porrum (leek), Allium cepa (onion) and Allium ursinum (bear's garlic, also known as bear paw garlic), particularly the latter two, whereby Allium cepa is particularly preferred. Examples of members of the species Allium cepa are common onions (with red or white or yellow skins) or shallots, whereby red or white common onions are preferred.

The extract containing the active ingredient of the invention, e.g. γ-L-glutamyl-trans-S-1-propenyl-L-cysteine sulfoxide, may be used in liquid form, for example in aqueous form, or in solid form, for example in granulate or powder form. Alternatively, the active ingredient of the invention, e.g. γ-L-glutamyl-trans-S-1-propenyl-L-cysteine sulfoxide, may be used as such, e.g. in solid, for example in powder or granulate form, or dissolved or dispersed in a liquid, e.g. in an aqueous liquid.

The amount of the active ingredient of the invention, e.g. γ-L-glutamyl-trans-S-1-propenyl-L-cysteine sulfoxide, to be supplied may vary within wide ranges, depending on i. a. the desired treatment, subject to be treated, e.g. human or a animal, and his needs. Thus, where the subject to be treated is an adult person (typically of ca. 60 to 75 kg body weight), a satisfactory inhibitory effect on bone resorption may, in general, be obtained with compositions formulated to allow a daily administration from about 20 to about 100 mg/kg, for example from about 40 to about 80 mg/kg of the active ingredient of the invention, e.g. γ-L-glutamyl-trans-S-1-propenyl-L-cysteine sulfoxide, e.g. on a solvent-free basis.

According to the invention, the term “nutritional compositions” refers to nutritional formulations and nutritional products, adapted for humans or animals, e.g. for mammals. The nutritional compositions according to the invention may be in the form of e.g. nutraceuticals, complete formula diet, nutritional or dietary supplements, such as animal feed supplement, functional food, beverage products, meal replacement, or food additives.

Suitable nutritional compositions, e.g. animal feed supplements, comprising the active ingredient of the invention, e.g. γ-L-glutamyl-trans-S-1-propenyl-L-cysteine sulfoxide, represent a further object of the invention. Accordingly, in one aspect the present invention provides a nutritional composition, e.g. an animal feed supplement, comprising:
(a) the active ingredient of the invention, e.g. γ-L-glutamyl-trans-S-1-propenyl-L-cysteine sulfoxide,
(b) a calcium source, and
(c) at least one energy source selected from the group consisting of carbohydrate, fat and nitrogen sources, and optionally
(d) Vitamin D.

Regarding component (a), the definitions, preferences and amounts given before for γ-L-glutamyl-trans-S-1-propenyl-L-cysteine sulfoxide apply. The nutritional compositions of the invention, e.g. animal feed supplements, conveniently comprise an amount of component (a) to allow a daily administration from about 20 to about 100 mg/kg, for example from about 40 to about 80 mg/kg.

The calcium source (b) may comprise any physiological acceptable inorganic or organic compound containing calcium. Examples are inorganic calcium salts, for example calcium chloride, calcium phosphate, calcium sulfate, calcium oxide, calcium hydroxide or calcium carbonate, or organic calcium components like whole or skim milk powder, calcium caseinate or calcium salts of organic acids such as calcium citrate, calcium maleate, or mixtures thereof. The use of organic calcium compounds, particularly skim milk powder, calcium caseinate or mixtures thereof, as calcium source (b) is preferred. The amount of calcium component to be supplied may vary within wide ranges. In general, the inventive compositions comprise in one unit dosage from about 100 mg to 1000 mg, preferably 200 mg to 700 mg and most preferred 300 to 600 mg of calcium (on an elemental basis).

The nutritional compositions of the invention, e.g. animal feed supplements, conveniently comprise for example from approximately 1 to 60% by weight, preferably from approximately 5 to 50% by weight and most preferred from 10 to 40% by weight of calcium component (b).

Suitable carbohydrate sources include for example maltodextrins, starch, lactose, glucose, sucrose, fructose, xylit and/or sorbit. In these forms the carbohydrates are both energy suppliers and sweeteners. The inventive compositions may contain one or more different carbohydrate sources.

Suitable fat sources include omega-6 polyunsaturated fatty acid sources, omega-3 polyunsaturated fatty acid sources, mono-unsaturated fatty acid sources, medium chain fatty acid sources (i.e. C₆-C₁₂-fatty acids); or mixtures thereof. The above-mentioned fatty acids may be employed in each case in form of the free acid, in mono-, di- or particularly in triglyceride form, or in form of a pharmacological or nutritional acceptable natural source.

Suitable natural sources of omega-6 polyunsaturated fatty acids include vegetable oils such as safflower oil, sunflower oil, soya oil, cotton oil and corn oil. Suitable natural sources of omega-3 polyunsaturated fatty acids include linseed oil and fish oils such as menhaden oil, salmon oil, mackerel oil, tuna oil codliver oil and anchovy oil.

Suitable natural sources of mono-unsaturated fatty acid sources are particularly omega-9 mono-unsaturated fatty acids, for example olives, canola, safflower (hybrids) and sunflower (hybrids).

A preferred fat source comprises triglyceride oils supplying the desired amounts of omega-6 polyunsaturated fatty acids and omega-3 polyunsaturated fatty acids and which are rich in the medium chain fatty acid residues (i.e. residues of C₆-C₁₂ fatty acid) and/or mono-unsaturated fatty acid residues. The inventive compositions, e.g. animal feed supplements, may contain one or more different fat sources.

Examples of suitable nitrogen sources of the inventive nutritional compositions, e.g. animal feed supplements, include sources containing nutritionally acceptable proteins such as soy bean derived proteins; milk proteins such as whey proteins or caseinates; and/or protein hydrosylates; and/or essential amino acids mixtures in free amino acid form or salt form; and/or compounds associated with the synthesis of polyamines, such as arginine, arginine precursors, ornithine and the like, in free amino acid form or salt form.

Preferred nitrogen sources of the nutritional compositions, e.g. animal feed supplements, are
(i) soy bean derived proteins, which may be employed in the form of soy beans or in the form of any suitable soya extract or concentrate, for example in form of soy flour, dried soy sprouts, soybean milk, or as dried aqueous extract from soybeans; or
(ii) milk proteins, for example whey derived proteins or caseinates which may be employed for example in the form of whey powder, caseinate salts such as calcium caseinate and/or whole or preferably skim milk powder and/or
(iii) a mixture of essential amino acids and/or
(iv) arginine as nitrogen source.

Milk proteins such as whey powder, caseinates, particularly calcium caseinate, and/or skim milk powder are another particularly preferred nitrogen source of the claimed nutritional compositions. The inventive compositions, e.g. animal feed supplements, may contain one or more different nitrogen sources.

The nutritional compositions, e.g. animal feed supplements, comprise for example, from approximately 0.1% to 98.9% by weight, preferably from approximately 1 to approximately 95% by weight, and most preferred from 10 to 90% by weight of energy source component (c).

The contribution of the nitrogen source, carbohydrate source and fat source to the caloric of the inventive nutritional compositions, e.g. animal feed supplements, may vary within wide ranges. For example, the carbohydrate source provides for 30 to 70% of the total energy supply, the nitrogen source for 5 to 45% and the fat source for 0.1 to 15% of the total energy supply of the composition. In preferred compositions of the invention the carbohydrate source provides for 40 to 60% of the total energy supply, the nitrogen for 20 to 35% and the fat source for 3 to 12% of the total energy supply of the composition.

A preferred energy source (c) of the inventive compositions, e.g. animal feed supplements, comprises

30 to 70% of the total energy supply of one or more carbohydrate sources selected from the group consisting of maltodextrins, starch, lactose, glucose, sucrose, fructose, xylit and sorbit;
5 to 45% of the total energy supply of one or more nitrogen sources selected from the group consisting of soy bean derived proteins, milk proteins, a mixture of essential amino acids and arginine and
0.1 to 15% of the total energy supply of one or more fat sources comprising omega-3- and omega-6-polyunsaturated fatty acids.

A particularly preferred energy source (c) of the inventive compositions comprises

40 to 60% of the total energy supply of one or more carbohydrate sources selected from the group consisting of maltodextrins, starch, lactose, glucose, sucrose, fructose, xylit and sorbit;
20 to 35% of the total energy supply of one or more nitrogen sources selected from the group consisting of soy bean derived proteins, skim milk powder and caseinates; and
3 to 12% of the total energy supply of one or more fat sources comprising omega-3- and omega-6-polyunsaturated fatty acids.

The amount of Vitamin D (optional component (d)) to be supplied may vary within wide ranges. In general, the inventive compositions comprise in one unit dosage from about 400 IU to 1000 IU, preferably about 500 IU.

The nutritional formulations of the invention, e.g. animal feed supplements, may comprise other nutritionally acceptable components such as vitamins, minerals, trace elements, fibers (preferably soluble fibers), flavors, preservatives, colorants, sweeteners, emulsifiers and the like.

Examples of vitamins suitable for the incorporation in the composition of the invention, e.g. animal feed supplements, include Vitamin A, Vitamin D, Vitamin E, Vitamin K, Vitamin C, folic acid, thiamin, riboflavin, Vitamin B₆, Vitamin B₁₂, niacin, biotin and panthotenic acid in pharmaceutical or nutritionally acceptable form.

Examples of mineral elements and trace elements suitable for the incorporation in the composition of the invention, e.g. animal feed supplements, include sodium, potassium, phosphorous, magnesium, copper, zinc, iron, selenium, chromium and molybdenum in pharmaceutical or nutritionally acceptable form.

The term soluble fiber as used herein refers to fibers which are able to substantially undergo fermentation in the colon to produce short chain fatty acids. Examples of suitable soluble fibers include agar-agar, alginates, carubin, carrageenan, gum arabic, guar gum, karaya gum, locust bean gum, pectin, tragacanth, or xanthan gum. They may be hydrolysed or not.

Suitable flavors include natural or artificial flavors, for example fruit flavors such as banana, orange, peach, pineapple or raspberry; vegetable flavors; or vanilla, cocoa, chocolate, coffee and the like.

Preferred ingredients of the inventive nutritional compositions, e.g. animal feed supplements, in addition to components (a), (b), (c) and (d) comprise beta-carotene (Vitamin A), Vitamin E, Vitamin C, thiamin, Vitamin B₁, B₆ and/or B₁₂, potassium, magnesium, selenium, zinc, phosphorous and soluble fiber in pharmaceutical or nutritionally acceptable form.

The nutritional compositions, e.g. animal feed supplements, may comprise for example, from approximately 0.1% to 15% by weight, preferably from approximately 0.2 to approximately 10% by weight, and most preferred from 0.5 to 5% by weight of these additional components other than components (a), (b), (c) and optionally (d).

The inventive nutritional formulations, e.g. animal feed supplements, may be formulated and administered in any form suitable for enteral administration, for example oral administration or tube feeding, e.g. nasal administration. The formulations are conveniently administered in the form of an aqueous liquid. The formulations suitable for enteral application are accordingly preferably in aqueous form or in powder or granulate form, whereby the powder or granulate is conveniently added to water prior to use. For use as tube feeding, the amount of water to be added will i.a. depend on the patient's fluid requirements and condition.

The inventive nutritional compositions, e.g. animal feed supplements, may be in form of a complete formula diet (in liquid or powder form), such that, when used as sole nutrition source essentially all daily caloric, nitrogen, fatty acids, vitamin, mineral and trace element requirements are met. In general, the daily amount to be supplied to adult persons will lie in the range of 750 to 3500 kcal/day, in particular of 1000 to 2000 kcal/day. However, the inventive nutritional compositions are preferably intended for use as a dietary supplement. The amount of energy supplied by a supplement should not be too excessive, in order not to unnecessarily suppress the patients appetite. The supplement conveniently comprises energy sources in an amount supplying from 50 to 1500 kcal/day, preferably 100 to 900 kcal/day and most preferred 150 to 700 kcal/day.

The nutritional compositions of the invention, e.g. animal feed supplements, which are in liquid form, for example in drink form, or in solid form, for example in granulate or powder form, may be obtained in a manner known per se, e.g. by admixing the ingredients and optionally adding water.

The invention further relates to pharmaceutical compositions, e.g. animal medicines or veterinary compositions, in single unit dose form comprising
(a) the active ingredient of the invention, e.g. γ-L-glutamyl-trans-S-1-propenyl-L-cysteine sulfoxide, and
(b) a pharmaceutical acceptable carrier, e.g. a carrier physiologically well tolerated by animals.

These pharmaceutical compositions, e.g. veterinary compositions, are compositions for enteral administration, such as oral, nasal or rectal administration. Suitable pharmaceutical compositions may be in liquid form or in solid form and comprise for example, from approximately 0.001% to 100% by weight, further example from approximately 0.1 to approximately 50% by weight, active ingredient (a).

Pharmaceutical compositions, e.g. veterinary compositions, for enteral administration are, for example, those in single unit dose forms, such as dragées, tablets, capsules or sachets. They are prepared in a manner known per se, for example by means of conventional mixing, granulating, confectioning, dissolving or lyophilising processes.

For example, pharmaceutical compositions for oral administration can be obtained by combining the active ingredient with solid carriers, optionally granulating a resulting mixture and processing the mixture or granules, if desired or necessary after the addition of suitable excipients, to form tablets or dragée cores.

Suitable carriers are especially fillers, such as sugars, for example lactose, saccharose, mannitol or sorbitol, cellulose preparations and/or calcium phosphates, for example tri-calcium phosphate or calcium hydrogen phosphate, and also binders, such as starch pastes using, for example, corn, wheat, rice or potato starch, gelatin, tragacanth, methylcellulose and/or polyvinylpyrrolidone, and, if desired, disintegrators, such as the above-mentioned starches, and also carboxymethyl starch, cross-linked polyvinylpyrrolidone, agar, or alginic acid or a salt thereof, such as sodium alginate. Excipients are especially flow-conditioners and lubricants, for example silicic acid, talc, stearic acid or salts thereof, such as magnesium or calcium stearate, and/or polyethylene glycol. Dragé cores are provided with suitable, optionally enteric, coatings, there being used inter alia concentrated sugar solutions which may contain gum arabic, talc, polyvinylpyrrolidone, polyethylene glycol and/or titanium dioxide, or coating solutions in suitable organic solvents or solvent mixtures or, for the preparation of enteric coatings, solutions of suitable cellulose preparations, such as acetylcellulose phthalate or hydroxypropylmethylcellulose phthalate. Dyes or pigments may be added to the tablets or dragé coatings, for example for identification purposes or to indicate different doses of active ingredient.

Other orally administrable pharmaceutical compositions are hard gelatin capsules and also soft, sealed capsules consisting of gelatin and a plasticiser, such as glycerol or sorbitol. The hard gelatin capsules may comprise the active ingredient in the form of granules, for example in admixture with fillers, such as lactose, binders, such as starches, and/or glidants, such as talc or magnesium stearate, and, if desired, stabilisers. In soft capsules the active ingredient is preferably dissolved or suspended in suitable liquids, such as fatty oils, paraffin oil or liquid polyethylene glycols, it is likewise being possible to add stabilisers.

Suitable rectally administrable pharmaceutical compositions are, for example, suppositories that consist of a combination of the active ingredient with a suppository base material. Suitable suppository base materials are, for example, natural or synthetic triglycerides, paraffin hydrocarbons, polyethylen glycols or higher alkanols. It is also possible to use gelatin rectal capsules which comprise a combination of the active ingredient with a base material.

Suitable base materials are, for example, liquid triglycerides, polyethylenglycols or paraffin hydrocarbons.

The inhibitory effect of the active ingredient of the invention, e.g. γ-L-glutamyl-trans-S-1-propenyl-L-cysteine sulfoxide on bone resorption, may be assessed by an in vitro assay, e.g. as described hereinabove, in which ivory slices, onto which freshly isolated osteoclasts have been settled, are incubated with a medium containing the extract or concentrate to be tested. The inhibitory effect on osteoclasts is assessed by counting the osteoclast resorption pits on the ivory slice.

The following Examples illustrate the invention.

### EXAMPLE 1

### Bioassay Guided Fractionation

1.1 To obtain a hydrophilic onion fraction, fraction A, adsorption column chromatography is used. The column is slurry-filled using aqueous 85% ethanol. After filling, the stationary phase (Amberlite XAD-4) is stepwise washed with 400 ml ethanol, 500 ml aqueous 85% ethanol, 2500 ml water and 500 ml aqueous 85% ethanol. After washing, the stationary phase is conditioned with the first solvent used for separation, i.e. aqueous ethanol 15% (v/v).

38.10 g of dry onion powder are dissolved in 600 ml aqueous ethanol 15% (v/v) and heated (60° C.) for 30 min under constant stirring. After cooling to room temperature, the turbid solution is centrifuged for 20 min at 7000 rpm and the supernatant is subjected to fractionation. The residue is discarded.

The pooled 15% aqueous fractions devoid of flavonoids, e.g. quercetin, and the 85% aqueous ethanolic fraction containing the flavonoids are reduced under vacuo at 40° C. and freeze-dried. The water fraction is discarded.

1.2 In order to separate saccharides such as fructose, glucose and sucrose from the active constituents of fraction A, preliminary reversed-phase high performance thin layer chromatography (RP-HPTLC) experiments, using 5% aqueous methanol as mobile phase, was performed. The method is upscaled on a reversed-phase medium pressure liquid chromatography (RP-MPLC) column and 1.0 g samples of fraction A are subjected to separation. Fractionation monitoring is performed by TLC using anisaldehyde reagent for detection. In order to recover as much starting material as possible, after each run, the column is thoroughly washed with methanol.

In order to isolate the bone resorbing inhibitory compound(s) of the hydrophilic onion fraction, fraction A, a bioassay guided fractionation is performed. To assess in vitro the bone resorbing inhibitory activity, the osteoclast pit assay is used. Medium containing 30 mg or less of freeze-dried fraction per ml is added to osteoclasts of new-born rats settled on ivory slices. After 24 hours of incubation, the tartrate-resistant acid phosphatase positive multi-nucleated cells, i.e. osteoclasts, are counted. Subsequently, the number of resorption pits is determined. Activity is calculated as the ratio of resorption pits per osteoclast and compared to a negative control, i.e. medium containing 10% fetal bovine serum and to a positive control, i.e. 10⁻¹² M calcitonin. For the analysis of statistical significance, the ratios of the treated groups±their respective SEMs are compared to the 95% confidence interval of the SEM of the negative control.
a) TLC-Screening and Yields of the RP-MPLC Fractionations.

The fraction containing the saccharides is named “fraction A2”, the fraction devoid of saccharides “fraction A1”.

| Fraction | Yields (g) | Yields (%) |
|---|---|---|
| A1 | 4.75 | 36.5 |
| A2 | 7.18 | 55.2 |
| Total | 11.93 | 91.7 |

b) Biological Results of the RP-M PLC Fractionations

Doses are given in mg per ml and results as resorption pits per tartrate resistant acid phosphatase positive (TRAP+)cells±SEM.

| Sample | Pits/TRAP+ cells ± SEM |
|---|---|
| Neg. control | 0.489 ± 0.128 |
| CT-10⁻¹²M | 0.061 ± 0.063 |
| 30 mg fraction A | 0.021 ± 0.021 |
| 12 mg fraction A1 | 0.243 ± 0.113 |
| Neg. control | 0.957 ± 0.327 |
| 24 mg fraction A1 | 0.144 ± 0.066 |
| Neg. control | 0.251 ± 0.071 |
| 30 mg fraction A | 0.015 ± 0.009 |
| 30 mg fraction A1 | 0.035 ± 0.016 |
| Neg. control | 1.210 ± 0.254 |
| 30 mg fraction A1 | 0.020 ± 0.009 |
| 30 mg fraction A2 | 0.399 ± 0.133 |
| 30 mg fraction A2 | 0.384 ± 0.124 |
| 30 mg fraction A | 0.115 ± 0.048 |

c) Discussion and Conclusion

Fraction A1 does not inhibit significantly osteoclast activity tested at the 1-fold proportional amount (12 mg/ml). However, the double dose (24 mg/ml) decreases osteoclast activity significantly to a pits/cells ratio of 0.144 (−40% compared to 12 mg/ml) and at 30 mg/ml even stronger inhibitions of osteoclast activity, i.e. 0.015 and 0.020 pits/cells (−90% compared to 12 mg/ml) can be measured. It is concluded that fraction A1 contains compounds inhibiting osteoclast activity.

The slight osteoclast activity inhibition by fraction A2 may be explained by the presence of small amounts of compounds of fraction A1.

Fraction A1 is chosen to continue the bioassay guided fractionation, fraction A2 is discarded. Because the bone resorbing inhibitory compound eluted still with the saccharides, an additional fractionation to separate the saccharides from the active compound(s) is performed.

1.3 Four different mobile passes for NP-TLC of polar compounds or sugars are tested in order to select the appropriate method for the next preparative separation. To evaluate the separation efficiency of the system, fraction A1 and the saccharides fructose, glucose and sucrose are used as samples:
(a) methylethylketone-acetic acid-methanol, 6:5:3 (v/v),
(b) acetone-water-hydrochloric acid 37%, 9 ml:1 ml:1 drop,
(c) n-butanole-acetic acid-diethylether-water, 9:6:3:1 (v/v),
(d) chloroform-methanol-water, 6.4:5:1.

All TLCs are performed on NP-TLC and sprayed with Anisaldehyde reagent for visualization. There are no remarkable qualitative differences between the mobile phases used. Mobile phase (d) was chosen to perform the next preparative separation step.

The NP-TLC system described above is upscaled to a NP-MPLC column and samples of 400 mg of fraction A1 are subjected to fractionation. Fractionation monitoring is again performed by NP-TLC. When no more spots are observed on TLC, the system is thoroughly washed with 70% aqueous methanol (v/v).
a) TLC-Screening and Yields of the NP-MPLC Fractionations

| Fraction | Yields(mg) | Yields (%) |
|---|---|---|
| A1-1 | 0.562 | 28.1 |
| A1-2 | 0.578 | 28.9 |
| A1-3 | 0.352 | 17.6 |
| A1-4 | 0.146 | 7.3 |
| Total | 1.638 | 81.9 |

b) Biological Results of the NP-MPLC Fractionations

Doses are given in mg per ml and results as resorption pits per tartrate resistant acid phosphatase positive (TRAP+) cells±SEM. In order to counteract losses during the fractionation, except for fraction A1-2, all fractions are tested at the one-, two- and three-fold proportional amount of their respective yields compared to fraction A.

| Sample | Pits/TRAP+ cells ± SEM |
|---|---|
| Neg. control | 0.990 ± 0.168 |
| 4.57 mg fraction A1-1 | 0.642 ± 0.207 |
| 9.88 mg fraction A1-1 | 0.809 ± 0.342 |
| 13.7 mg fraction A1-1 | 0.691 ± 0.147 |
| Neg. control | 0.957 ± 0.327 |
| 5.28 mg fraction A1-2 | 0.835 ± 0.231 |
| 15.8 mg fraction A1-2 | 1.000 ± 0.230 |
| Neg. control | 0.818 ± 0.141 |
| 2.15 mg fraction A1-3 | 0.631 ± 0.186 |
| 4.64 mg fraction A1-3 | 0.569 ± 0.277 |
| 6.44 mg fraction A1-3 | 1.425 ± 0.396 |
| Neg. control | 1.419 ± 0.364 |
| 1.48 mg fraction A1-4 | 2.158 ± 0.888 |
| 3.12 mg fraction A1-4 | 3.636 ± 1.262 |
| 4.76 mg fraction A1-4 | 0.010 ± 0.010 |
| 4.76 mg fraction A1-4 | 0.096 ± 0.026 |

c) Discussion and Conclusion

In fractions A1-1, A1-2 and A1-3 no inhibition is measurable. The pits per cell ratio of all these fractions are located inside the 95% confidence interval of the SEM of the negative control. Fraction A1-4, completely free of sugars, shows a significant osteoclast activity inhibition a the three-fold dose. The apparent stimulation of the cell activity at the one- and two-fold dose may be explained by a strong decrease in cell number. Fraction A1-4 is chosen for further fractionation.

1.4 Fractionation of Fraction A1-4 by Semi-Preparative Reversed-Phase HPLC (SP-RP-HPLC)

Fraction A1-4 is further fractionated with SP-RP-HPLC into four fractions A1-4A, A1-4B, A1-4C and A1-4D using as solvent an isocratic water/acetonitrile system buffered with 0.00625% formic acid. Fraction A1-4B contains two minor compounds of fraction A1-4, fraction A1-4C consists of the most predominant compound of fraction A1-4, fractions A1-4A and A1-4D are the prerun and afterrun. Fractions A1-4A and A1-4D are pooled together for the further tests. On the whole, 8.125 mg of fraction A1-4 are separated in 65 single HPLC runs applying 0.125 mg in each run and pooling the fractions. Fractionation is performed manually by switching a tap at the outlet of the HPLC equipment.
a) Yield of the SP-RP-HPLC Fractionations

| Fraction | Yields (mg) | Yields (%) |
|---|---|---|
| A1-4A | 3.7 | 35.2 |
| A1-4B | 1.3 | 12.5 |
| A1-4C | 1.6 | 15.2 |
| A1-4D | 3.9 | 37.1 |
| Total A1-4A-A1-4D | 10.5 | 100.00 |

b) Biological Results of the SP-RP-HPLC Fractionations

| Sample | Pits/TRAP+ cells ± SEM |
|---|---|
| Neg. control | 0.739 ± 0.138 |
| CT 10⁻¹²M | 0.116 ± 0.030 |
| 2.28 mg A1-4 | 0.193 ± 0.055 |
| 2.53 mg A1-4A + D | 0.432 ± 0.184 |
| 0.43 mg A1-4B | 0.718 ± 0.208 |
| 0.53 mg A1-4C | 0.325 ± 0.108 |

c) Discussion and Conclusion

Fraction A1-4 at a three-fold proportional dose corresponding to fraction A1 and fraction A1-4C significantly (p<0.5) inhibit bone resorption. Fraction A1-4C inhibits osteoclast activity similar to fraction A1-4, indicating that A1-4C is the sole active compound of fraction A1-4. The isolation of A1-4C is repeated to recover sufficient amounts for structure elucidation experiments.

### SUMMARY

An ethanolic bulb extract is purified by sequential use of four different chromatographic systems—Amberlite XAD-4 extraction; RP-M PLC; NP-MPLC; and semi-preparative RP-HPLC. Resulting fraction A1-4C consists of only one compound inhibiting osteoclast activity.

The following graph gives an overview of the bioassay-guided isolation leading to fraction A1-4C:

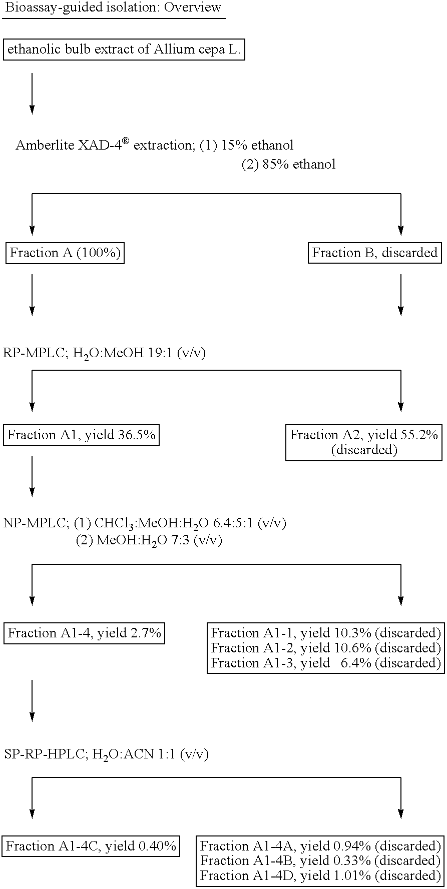

### EXAMPLE 2

### Structure Elucidation Experiments

2.1 RP-HPLC-ESI-MS² of A1-4C

Mass spectroscopical analysis is performed using HPLC-ESI-MS equipment to obtain first structural information concerning the structure of A1-4C. The MS is equipped with a quadrupole ion trap. Fragmentation is achieved by colliding the positively charged, ionized molecule with helium gas using a collision energy of 35%.

The positively charged parent ion of A1-4C is 307 m/z [m+H]⁺. Literature search revealed the existence of an onion compound of identical mass, namely γ-L-glutamyl-trans-S-1-propenyl-L-cysteine sulfoxide (g-GPeCSO). Further fragmentation is performed to see whether the resulting fragmentation pattern can be brought into line with this compound.

2.2 ESI-MS of A1-4C After Acid Hydrolysis

The results obtained by ESI-MS further support the assumption that fraction A1-4C is g-GPeCSO and are not contradictory to the previously established claim after the HPLC-ESI-MS experiments.

2.3 Nuclear Magnetic Resonance Experiments of A1C-4C

Measurements are performed in D₂O using trimethylsilyl-propansulfonic acid as external standard.

The compound of fraction A1-4C can be identified by NMR spectroscopy as γ-L-glutamyl-trans-S-1-propenyl-L-cysteine sulfoxide (g-GPeCSO), confirming the preceeding HPLC-MS experiments.

### EXAMPLE 3

### Bioassay Guided Fractionation—Scaled Up Isolation

3.1 Extraction. Dried flakes of onion (Allium cepa L., Liliaceae) were pulverized and 400 g extracted twice with 2000 mL of aqueous ethanol (85% v/v) at 60° C. for 1 h and filtered. After evaporation of the ethanol the aqueous residue was freeze-dried and stored at −20° C.

3.2 Bioassay-Guided Fractionation. Fractions A and B. About 38 g of the onion extract were redissolved in 600 mL of aqueous ethanol 15% (v/v) and heated at 60° C. for 30 min under constant stirring. After cooling to room temperature, the mixture was centrifuged for 20 min at 7000 r.p.m. and the supernatant subjected to fractionation by adsorption column chromatography (residue discarded). Fraction A and fraction B resulted by using Amberlite XAD-4 (Fluka Chemie, Buchs, Switzerland) as stationary phase and eluting with (1) 1280 mL of aqueous ethanol 15%, (2) 1280 mL of water and (3) 1400 mL of aqueous ethanol 85% at a flow of 10 mL/min. Thin-layer chromatography (TLC) on Silicagel 60 F₂₅₄ plates 10×10 cm (Merck, Darmstadt, Germany) with chloroform-methanol-water 6.4:5:1 (v/v) as mobile phase and visualizing with a 1% methanolic solution of diphenylboric acid-β-ethylaminoate, followed by a 5% ethanolic solution of polyethyleneglycole 4000, showed the hydrophilic fraction A completely devoid of flavonoids, whereas the lipophilic fraction B contained flavonoids. Fraction A and B were then tested in vitro and in vivo by using the osteoclast pit assay and the urinary [³H]-tetracycline excretion rat model, respectively, described further below. Fraction A showed bone resorption-inhibiting effects, whereas fraction B was inactive and therefore discarded.

Fractions A 1 and A 2. 1 g-aliquots of fraction A, dissolved in 5 mL of mobile phase, were then further separated by medium-pressure liquid chromatography (MPLC; 681 pump, 684 fraction. collector, both from Büchi, Flawil, Switzerland) on Lichroprep RP-18, 15-25 μm (Merck, Darmstadt, Germany), 46×2.5 cm with 13×1 cm pre-column; elution was with (1) 340 mL of aqueous methanol 5%, and (2) 300 mL of methanol at a flow rate of 4 mL/min. The monitoring of the 60 4-mL fractions was performed by high-performance thin layer chromatography (HPTLC) on RP-18 F₂₅₄ 10×10 cm plates (Merck, Darmstadt, Germany) with water-methanol 19:1 as mobile phase and anisaldehyde reagent (5% acetic acid solution of anisaldehyde followed by heating at 120° C.) for visualization. Fraction A1 contained mainly saccharides (glucose, fructose, sucrose) and was active in vitro, fraction A2 was free of saccharides, almost inactive and therefore discarded.

Fractions A 1-1, A 1-2, A 1-3, A 1-4. 400 mg-aliquots of fraction A1 were further separated by MPLC on Silicagel 60, 15-40 μm (Merck, Darmstadt, Germany), 22×2.5 cm with 13×1 cm pre-column; elution was with (1) 1208 mL of chloroform-methanol-water 6.4:5:1, and (2) 300 mL of methanol at a flow rate of 4 mL/min. The monitoring of the 120 8-mL fractions was performed by TLC on silicagel 60 F₂₅₄ 10×10 cm plates (Merck, Darmstadt, Germany) with n-butanol-n-propanol-acetic acid-water 3:1:1:1 as mobile phase and anisaldehyde reagent for visualization. The now saccharides-free fraction A 1-4 showed a significant in vitro activity, the saccharides-containing fractions A 1-1, A 1-2, and A 1-3 were not active in the osteoclast pit assay and therefore not further studied.

Fractions A 1-4A, A 1-4B, A 1-4C, A 1-4D. Aliquots of 0.125 mg of fraction A 1-4 were then finally purified by semipreparative, isocratic high performance liquid chromatography (HPLC; HP 1090 Liquid Chromatograph with Diode Array Detection (DAD), Hewlett-Packard, Waldbronn, Germany) on a 250×10 mm i.d. Spherisorb ODS-1 5 μm column at 40° C. The mobile phase was water-acetonitrile 1:1, containing 0.00625% formic acid at a flow rate of 1.5 mL/min. Detection was at 195 nm. Equal fractions were pooled, evaporated and freeze-dried and tested in vitro. Fraction A 1-4C, corresponding to the major HPLC peak and chromatographically pure, inhibited osteoclast activity nearly as strong as fraction A 1-4, all other fractions were inactive.

3.3 Scaled Up Isolation of A 1-4C. For the preparation of larger amounts of A 1-4C (GPCS) 10.8 g-aliquots of fraction A, dissolved in water, were first separated by cation exchange column chromatography on a 43×3 cm column filled with Dowex 50WX8 (Fluka Chemie, Buchs, Switzerland). The non-cationic fraction eluted with 975 mL of water, and the cationic fraction with 1425 mL of 0.75 M ammonia at a flow rate of 15-20 mL/min. TLC on silicagel 60 F₂₅₄ with n-butanol-n-propanol-acetic acid-water 3:1:1:1 as mobile phase, and acetaldehyde and ninhydrin reagent for visualization were used for the fraction monitoring. In addition, the fractions were checked by HPLC-DAD, using a 125×4 mm Spherisorb ODS-1 3 μm column, water-acetonitrile 1:3, containing 0.5% phosphoric acid at a flow of 0.7 mL/min as mobile phase, and detection at 195 nm. The non-cationic fraction contained mainly saccharides, whereas in the cationic fraction an enrichment of peptides and amino acids could be observed. Consequently, 700 mg-aliquots of the freeze-dried cationic fractions were further separated by anion exchange column chromatography on a 400×26 mm column filled with Dowex 1X8 (Fluka Chemie, Buchs, Switzerland). 237.5 mL of 0.1 M acetic acid, 1250 mL of 0.5 M acetic acid, and 1000 mL of 2 M acetic acid were used for elution, the flow rate of 95 mL/h maintained by a pericyclic pump Minipuls 3 (Gilson, Villiers-le-Bel, France) and the fractions collected by a 7000 Ultrorac (Amersham Biosciences, Dübendorf, Switzerland) and monitored by TLC (see above). Equal fractions were pooled and freeze-dried prior to a final check by HPLC-ESI-MS and HPLC-DAD.

3.4 Structure Elucidation of Compound A 1-4C. HPLC-electrospray ionization-tandem mass spectrometry (HPLC-ESI-MS-MS). The instrumentation consisted of a HP 1100 Liquid Chromatograph with DAD (Hewlett Packard, Waldbronn, Germany), linked to a LCQ ESI mass spectrometer (Finnigan, Bremen, Germany). Separation was performed isocratically at 40° C. on a Spherisorb ODS-1 3 μm column, 125×4 mm (Macherey-Nagel, Düren, Germany) with water-acetonitrile 1:4 containing 0.5% formic acid at a flow rate of 0.7 mL/min. The collision gas was helium, the energy 35%. Measurements were performed both in the positive and negative ionization mode.

ESI-MS-MS. For the confirmation of the results obtained by HPLC-ESI-MS-MS, A 1-4C was further analyzed after acidic hydrolysis by 70% formic acid (100° C. for 22 h) by direct inlet ESI-MS-MS. The instrument was an Applied Biosystems/Sciex Qstar Pulsar Mass Spectrometer (Foster City, U.S.A.), which is a hybrid quadrupole time-of-flight (TOF) MS equipped with a nano-electrospray ion source.

3.5 Nuclear magnetic resonance spectroscopy (NMR). NMR experiments were performed on a Bruker DRX500 instrument (Karlsruhe, Germany). Experiments were run at 500 MHz for ¹H and 125 MHz for ¹³C. D₂O was purchased from Euriso-Top (Gif-sur-Yvette, France) and chemical shifts were reported relative to trimethylsilyl-propionic acid (Na-salt) as an external standard (Wilmad Corp., Buena, N.J., USA).

TABLE 3.5
NMR Data for GPCS in D₂O

| Position | . δ_{C} | . δ_{H} Multiplicity (J, Hz) |
|---|---|---|
| 1 | 21.0 | 1.95 dd (J₁₂ 6.87, J₁₃ 1.54) |
| 2 | 147.0 | 6.65 dq (J₂₁ 6.87, J₂₃ 15.26) |
| 3 | 133.0 | 6.50 dq (J₃₁ 1.54, J₃₂ 15.26) |
| 4 | 57.0 | 3.30 dd (J₅₄ 9.77, J_{55′} 13.28) |
| | | 3.45 dd (J_{5′4} 4.43, J_{5′5} 13.28) |
| 5 | 54.0 | 4.50 dd (J₄₅ 4.43, J_{45′} 9.77) |
| 6 | 178.5 | |
| 7 | 179.0 | |
| 8 | 35.0 | 2.50 t (J₈₇ 7.78) |
| 9 | 30.0 | 2.15 tt (J₇₆ 6.41, J₇₈ 7.78) |
| 10 | 57.0 | 3.80 t (J₆₇ 6.41) |
| 11 | 178.1 | |

Solvents for all HPLC experiments were of Lichrosolv® gradient grade quality, chemicals and solvents for MPLC and column chromatography as well as TLC detection reagents were of p.a. quality from Merck (Darmstadt, Germany).

3.6 Quantitative Determination of GPCS in Onion by HPLC. GPCS in onion was quantified using a HP 1090 Liquid Chromatograph with DAD set at 195 nm (Hewlett Packard, Waldbronn, Germany). Analysis was performed isocratically at 40° C. on a Spherisorb ODS-1 3 μm column, 125×4 mm (Macherey-Nagel, Duren, Germany) with water-acetonitrile 1:4 containing 0.5% formic acid at a flow rate of 0.7 mL/min. GPCS was extracted from dried, pulverized onion with methanol-water (50:50; v/v) at room temperature according to the methods described in Mütsch-Eckner, M.; Sticher, O.; Meier, B. Reversed-phase high-performance liquid chromatography of S-alk(en)yl-L-cysteine derivatives in Allium sativum including the determination of (+)-S-allyl-L-cysteine sulphoxide, g-L-glutamyl-S-allyl-L-cysteine and g-L-glutamyl-S-(trans-1-propenyl)-cysteine. J Chromatogr 1992, 625, 183-190. This method allowed an efficient extraction of polar compounds using hydrophilic solvents and at the same time inhibiting cleaving enzymes such as glutamylpeptidases and alliinases by the addition of methanol. The residues remaining after filtration were re-extracted twice to thoroughly extract GPCS. Finally, the methanol was removed from the filtrates in vacuo prior to freeze-drying and HPLC analysis.

The structure of this compound was elucidated with high performance liquid chromatography-electrospray ionization-mass spectrometry, time-of-flight electrospray ionization mass spectrometry, and nuclear magnetic resonance spectroscopy.

The single peak was identified as gamma-L-glutamyl-trans-S-1-propenyl-L-cysteine-sulfoxide and has a molecular weight of about 306 u.

3.7 Biological Testing. Animals. Wistar Hanlbm rats (RCC Ltd., Füllinsdorf, Switzerland) were reared and kept in standard animal facilities that comply with the Swiss and U.S. National Institutes of Health guidelines for care and use of experimental animals. The experiment performed was approved by the State Committee for the Control of Animal Experimentation. At completion of the experiment the rats were euphonized with carbon dioxide.

In vivo assessment of bone resorption. The urinary excretion of ³H-labeled tetracycline ([³H]-Tc) from chronically prelabeled rats, an extensively validated method, was used to assess bone resorption (3;4;8-11). For the present experiment 3 Wistar Hanlbm dams with 12 three-day-old male pups each were purchased. The 36 pups were injected from the first week of life twice a week for 6 weeks with increasing amounts of [³H]-Tc in accordance with the method described in Mühlbauer, R. C.: Fleisch, H. A method for continual monitoring of bone resorption in rats: evidence for a diurnal rhythm. Am J Physiol 1990, 259, R679-R689. [³H]-Tc is deposited into bone and is released when bone is resorbed (Am J Physiol 1990, 259, R679-R689). After discontinuation of labeling, the rats were transferred to metabolic cages. After 10 days of acclimatization baseline bone resorption was monitored by measuring the daily urinary [³H]-excretion. After 10 days of baseline measurement the 10-day dietary intervention was started in rats which were homogeneously assigned to the groups, that is, the baseline [³H] urinary excretion of all rats was ranked and to each treatment group one animal with a similar rank was assigned until the number of animals per group was completed (n=6 for the control group; n=5 per treatment group). Using this protocol the mean [³H]-excretion was similar for all groups at the start of the dietary intervention. ³H in urine was determined by liquid scintillation counting. Aliquots of 1 mL urine were counted in 10 mL of Irga-Safe Plus™ scintillator (Packard International, Zürich, Switzerland) and the result (dpm) was multiplied by the 24 h urine volume.

Feeding and Diet. From the time when the rats were housed in the metabolic cages, they were given demineralized water to drink. The diets were given in a stainless steel crucible as wet food to minimize spillage in the cage; thus, deionized water was added to batches of food powder to give a dough-like consistency which allowed to form food-balls. During the 10 days acclimatization period in the metabolic cages and during the 10 days baseline urine collection, the rats were fed a standardized “normal” diet 2134 (Kliba-Mühlen, Kaiseraugst, Switzerland) with a similar high Ca and P concentration (1.1 g Ca and 1.2 g P per 100 g) as used in the “semi-purified” diet described below. During the acclimatization period the rats were trained to consume 23 grams of wet food/day (13.1 g dry matter); rats which repeatedly did not eat the whole daily amount were eliminated during this period. For the dietary intervention the dry additives were mixed with a “semi-purified” diet (J Nutr 2003, 133, 3592-3597). Appropriate amounts of the items to be investigated were added to batches of wet food sufficient for feeding 5 rats during 10 days. These diets were then aliquoted into daily portions and kept frozen at −20° C. until use. The calcium and phosphate concentration of the diets was verified in triplicate ashed samples dissolved in 1 mol/L HCl. Calcium was determined by atomic absorption spectrophotometry and phosphate by photometry (8;13). The values given by the manufacturer were confirmed.

Bioassay (in vitro assessment of osteoclast activity). Osteoclasts were isolated from femora and tibiae of 2-day-old rats and settled for 40 min onto 4×4 mm ivory slices used as the mineralized substrate. After washing off non-adherent cells, individual slices were transferred to 48-well tissue culture plates and incubated for 24 h at 37° C. in a 5% CO₂/air atmosphere (14;15) in medium containing 10% fetal bovine serum (FBS) with or without the material to be tested. The concentration of bicarbonate in the MEM Earle's medium was reduced to 15 mM by addition of 12M HCl. In each experiment calcitonin (salmon calcitonin, Novartis Pharma, Basel, Switzerland) was added as positive control at the concentration of 10⁻¹¹ M or 10⁻¹² M. In one experiment parathyroid hormone (PTH; bovine 1-34, Bachem, Bubendorf, Switzerland) was added at the concentration of 10⁻⁸ M, in order to stimulate bone resorption. For each material to be tested 8 slices were used, for the untreated control 2×8 slices were used. After fixation osteoclasts were stained for tartrate-resistant acid-phosphatase (TRAP) (kit 386-A, Sigma, Buchs, Switzerland) and were counted blinded as TRAP positive (TRAP+) multinucleated (more than 2 nuclei) cells (MNC). After removing the cells, the slices were sputter coated with gold and the resorption pits counted blinded in a process described in Vitté, C.; Fleisch, H.: Guenther, H. L. Bisphosphonates induce osteoblasts to secrete an inhibitor of osteoclast-mediated resorption. Endocrinology 1996, 137, 2324-2333. Osteoclastic resorption activity is calculated as the ratio: resorption pits/TRAP+MNC.

### RESULTS AND DISCUSSION

Consecutive bioassay-guided chromatographic fractionations on nonionic-polymeric, reversed phase and normal phase columns of the ethanolic extract active in vivo and in vitro, resulted in fractions A (51.52% (w/w), average yield corresponding to prior fraction), A1 (36.48%) and A1-4 (7.34%), respectively. 10.5 mg of fraction A1-4 were purified by semipreparative RP-18 HPLC leading to 1.6 mg (15.20%) of fraction A1-4C (GPCS).

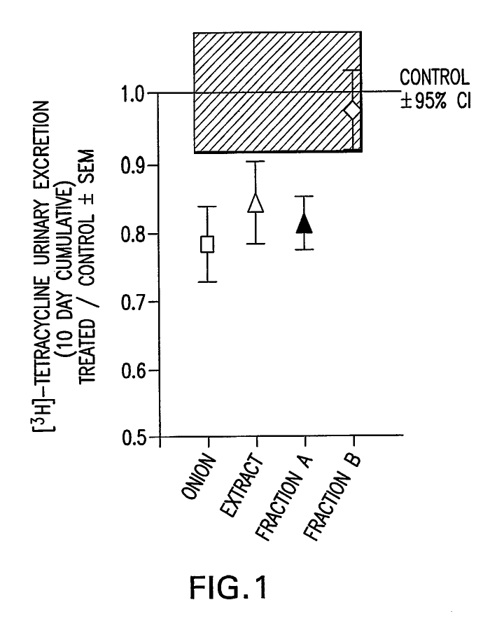

The bone resorption inhibitory activity from onion is associated with the polar fraction A (FIG. 1). On the contrary, fraction B containing flavonoids is devoid of activity when tested in vivo at a dose corresponding to 1 g of dry onion. This contrasts with the opinion of others who have proposed that rutin (described in Horcajada-Molteni, M. N.; Crespy, V.; Coxam, V.; Davicco, M. J.; Remesy, C.; Barlet, J. P. Rutin inhibits ovariectomy-induced osteopenia in rats. J Bone Miner Res 2000, 15, 2251-2258), a flavonoid abundant in onion, could be responsible for the effect of onion and other vegetables observed (Nature 1999, 401, 343-344). In that study a single pharmacological dose of rutin inhibited bone loss in rats, a dose which was, however, much higher than that contained in 1 g of vegetables (Nature 1999, 401, 343-344). Therefore, it is open whether rutin contributes to the inhibition of bone resorption in vivo (Mühlbauer, R. C. Rutin cannot explain the effect of vegetables on bone metabolism. (Letter to the Editor). J Bone Miner Res 2001, 16, 970; and Barlet, J. P. A possible rut(in) the road. (Reply). J Bone Miner Res 2001, 16, 971).

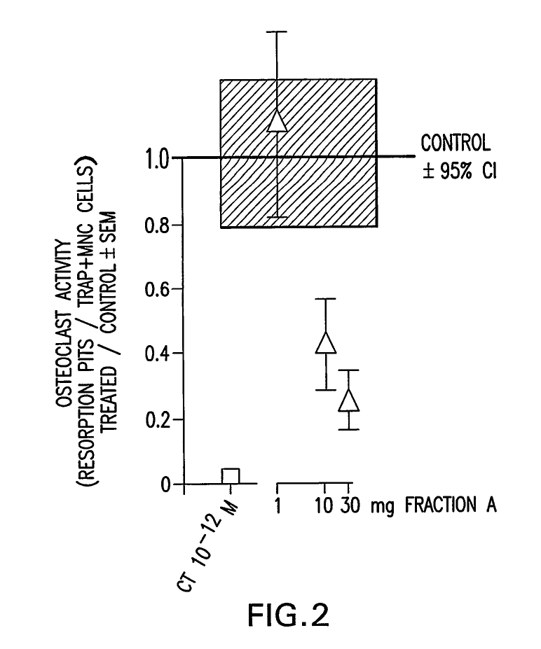

Fraction A also inhibits the resorption activity of osteoclasts in vitro (FIG. 2) when tested at doses corresponding to 1.74, 17.4 and 52.2 mg/mL of dry onion equivalents. Therefore, this in vitro model could be used as bioassay since it also requires only small amounts of material for activity testing. Fraction B was also investigated in this model (results not shown) at doses equivalent to 9, 17 and 26 mg/mL dry onion, i.e. 0.06, 0.12 and 0.18 mg/mL medium. At the higher concentrations the lipophilic material was cytotoxic (very low number of surviving cells and many fragments of necrotic cells), while at 9 mg/mL onion equivalents, a dose with only a negligible effect on cell number, we could not detect an effect. Thus, fraction B containing the onion flavonoids was considered also as in vitro inactive. This is in agreement with our additional evidence suggesting that rutin cannot explain the effect of vegetables on bone metabolism (J Bone Miner Res 2001, 16, 970).

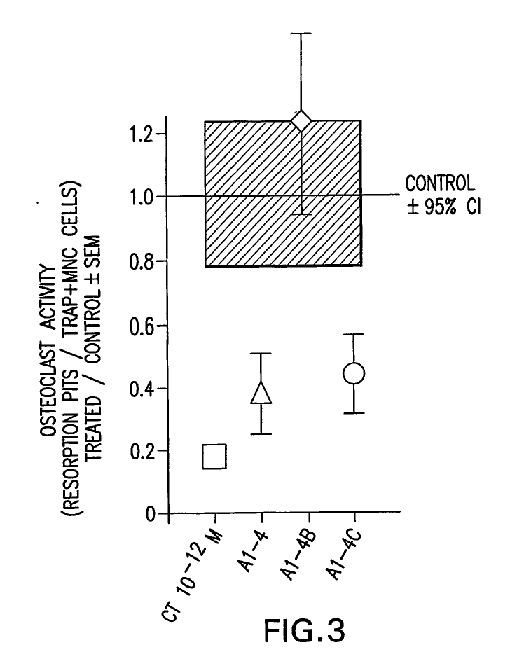

Further extensive isolation work by using semi-preparative HPLC and activity screening allowed to identify A 1-4C as the only active fraction derived from the starting material A 1-4 (FIG. 3). Furthermore, also the pooled HPLC peaks eluting before (fractions A 1-4A and A 1-4B) and after A 1-4C (fraction A 1-4D) were tested (2.53 mg/ml), but they were cytotoxic so that no conclusion could be drawn (results not shown). As HPLC showed that fraction A 1-4C consists of a single compound its subsequent identification was attempted.

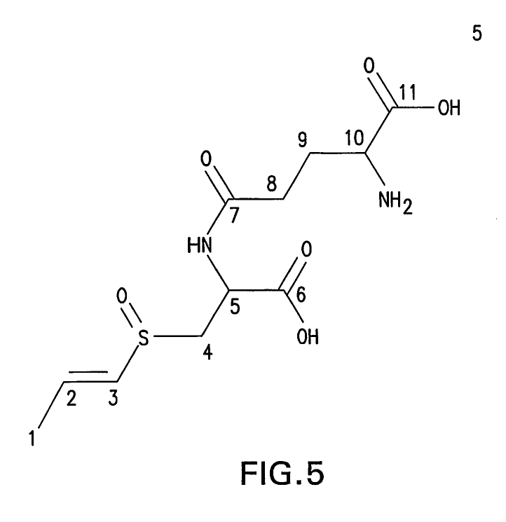

HPLC-ESI-MS-MS experiments with the compound A 1-4C showed a parent ion of m/z 307 and of m/z 305 in the positive and in the negative ionization mode, respectively. Thus, the uncharged molecular ion of the compound in fraction A1-4C was 306 u. A survey of the literature of onion compounds (Breu, W. Allium cepa L. (onion) Part 1: Chemistry and analysis. Phytomedicine 1996, 3, 293-306) revealed the compound to be gamma-L-glutamyl-trans-S-1-propenyl-L-cysteine-sulfoxide (GPCS; see FIG. 5). Moreover, an observed mass of m/z 130 fitted to the presence of a gamma-glutamyl-type bond in the molecule as described by other investigators (Isobe, M.; Uyakul, D.; Liu, K. L.; Goto, T. FAB-MS/MS spectrometry in determining the primary structure of g-glutamyl-containing peptides. Agric Biol Chem 1990, 54, 1651-1660.). In the ESI-MS-MS spectra after acid hydrolysis fragments corresponding to glutamic acid, cysteine, and cystine could be observed, thus confirming the hypothesis. ¹H-, ¹H/¹H COSY and ¹³C/¹H HSQC NMR measurements confirmed the presence of the two amino acids glutamic acid (C7, C8, C9, C10, and C11), cysteine (C4, C5, and C6), and an aliphatic C-chain with a double bond (C1, C2, and C3) in the molecule (FIG. 5). The coupling constant of 15.26 Hz between H2 and H3 indicated a trans configuration (Kuttan, R.; Nair, N. G.; Radhakrishnan, A. N.; Spande, T. F.; Yeh, H. J.; Witkop, B. The isolation and characterization of g-L-glutamyl-S-(trans-1-propenyl)-L-cysteine sulfoxide from sandal (Santalum album L). An interesting occurrence of sulfoxide diastereoisomers in nature. Biochemistry 1974, 13, 4394-4400). For chemical shifts and couplings see Table 1. Focussing on the ¹³C-carboxy-region in the ¹³C/¹H HMBC spectrum revealed couplings of the gamma-C7 of the glutamic acid (174.00 ppm) with the alpha-H5 of cysteine (4.50 ppm) and with the two gamma-H8 of glutamic acid (2.50 ppm) and a coupling of the alpha-C11 of the glutamic acid (173.10 ppm) only with the alpha-H10 of glutamic acid (3.80 ppm). This proves clearly the presence of a gamma-glutamyl-type bond in the molecule. These findings were confirmed by spectral comparison with a pure reference sample of GPCS (Shaw, M. L.; Lancaster, J. E.; Lane, G. A. Quantitative analysis of the major g glutamyl peptides in onion bulbs (Allium cepa). J Sci Food Agric 1989, 48, 459-467).

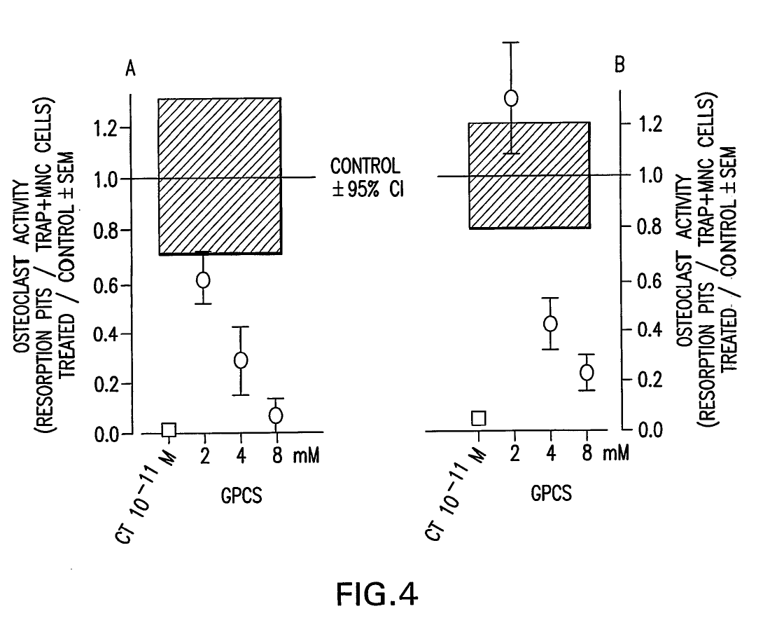

Up-scaled isolation of GPCS was carried out by subjecting 108.0 g of fraction A first to cation followed by anion exchange column chromatography yielding 6.974 g (6.46%) of cationic fraction and 788.0 mg of GPCS (0.73%), respectively. Fractions devoid of GPCS were discarded. GPCS isolated by this methodology inhibits the resorption activity of osteoclasts dose-dependently, irrespective of whether the osteoclasts are stimulated or not with PTH (FIG. 4). However, the magnitude of the inhibition is somewhat smaller when PTH is added to the cultures than without. This seems also to apply for the positive control calcitonin. Fraction A 1-4C was active at the concentration of 0.53 mg/mL, i.e. at 1.7 mM GPCS, while in the present experiment the inhibition was not significant at 2 mM. Taken together, the minimal effective dose of GPCS in this model appears to lay around 2 mM in cultures not stimulated with PTH. In cultures stimulated with PTH this value lays between 2 and 4 mM, a graphical interpolation from FIG. 4 suggests that it may lay around 3 mM.

As no other constituent of fraction A 1-4C displayed inhibitory activity, GPCS appears to be responsible for the effect of onion on bone resorption in vitro, a contention that should be confirmed in vivo as soon as sufficient compound is available. Future studies are also required to establish the doses necessary to inhibit bone loss in an osteoporosis model, and to study its mechanism of action on bone cells. According to the present literature, the compounds occurring in plant-derived foodstuffs which support bone health are limited to minerals (calcium, potassium and magnesium), to vitamins (K and C), to phytoestrogens (coumestrol, zearalenol, isoflavones and humulone), possibly to other flavonoids (rutin and hesperidin) and to monoterpenes (Horcajada-Molteni, M. N.; Crespy, V.; Coxam, V.; Davicco, M. J.; Remesy, C.; Barlet, J. P. Rutin inhibits ovariectomy-induced osteopenia in rats. J Bone Miner Res 2000, 15, 2251-2258; Tucker, K. L.; Hannan, M. T.; Chen, H.; Cupples, L. A.; Wilson, P. W.; Kiel, D. P. Potassium, magnesium, and fruit and vegetable intakes are associated with greater bone mineral density in elderly men and women. Am J Clin Nutr 1999, 69, 727-736; New, S. A.; Bolton-Smith, C.; Grubb, D. A.; Reid, D. M. Nutritional influences on bone mineral density: a cross-sectional study in premenopausal women. Am J Clin Nutr 1997, 65, 1831-1839; and Draper, C. R.; Edel, M. J.; Dick, I. M.; Randall, A. G.; Martin, G. B.; Prince, R. L. Phytoestrogens reduce bone loss and bone resorption in oophorectomized rats. J Nutr 1997, 127, 1795-1799; Arjmandi, B. H.; Alekel, L.; Hollis, B. W.; Amin, D.; Stacewicz-Sapuntzakis, M.; Guo, P.; Kukreja, S. C. Dietary soybean protein prevents bone loss in an ovariectomized rat model of osteoporosis. J Nutr 1996, 126, 161-167). Thus, compounds active on bone, and therefore candidates for a dietary approach to osteoporosis, are widely distributed in the plant kingdom.

We found 17.3 mg of GPCS in 1 g of dry onion, i.e. 1.73% (w/w). This result is in agreement with the findings of others who measured GPCS concentrations in the range of 0.58-2.88% of dry weight (Shaw, M. L.; Lancaster, J. E.; Lane, G. A. Quantitative analysis of the major □ glutamyl peptides in onion bulbs (Allium cepa). J Sci Food Agric1989, 48, 459-467; and Kopsell, D. A.; Randle, W. M. Selenium affects the S-alk(en)yl cysteine sulfoxides among short-day onion cultivars. J Am Soc Hortic Sci 1999, 124, 307-311).

The identification of GPCS as a compound inhibiting the activity of bone resorbing cells adds another compound, belonging yet to another class of molecules, to the list of natural compounds active on bone. Whether GPCS is a representative of a family of active compounds or an individual active compound is presently not known. To clarify this issue, it will be necessary to study its role in the activity of the other 25 active vegetable food items identified so far (J Nutr 2003, 133, 3592-3597), and possibly identify other active members of this class of compounds.

### EXAMPLE 4

The following is an example of a suitable composition of an inventive Supplement in powder form.

Supplement in Powder Form (1 portion)

| | | | |
|---|---|---|---|
| Content | | 65.0 | g |
| Extract¹⁾ | | 14.5 | g |
| Protein | | 20.0 | g |
| including | Ca-caseinate protein | 8.7 | g |
| | skim milk powder | 11.0 | g |
| Fat | | 2.8 | g |
| including | omega-6 polyunsaturated acids | 1.3 | g |
| | omega-3 polyunsaturated acids | 0.03 | g |
| Carbohydrates | (including inventive extract) | 31.0 | g |
| including | lactose | 16.5 | g |
| | maltodextrin | 3.5 | g |
| Fiber (soluble) | | 5.0 | g |
| Further ingredients | | 3.0 | g |
| including | Na | 230 | mg |
| | K | 500 | mg |
| | Ca | 600 | mg |
| | Mg | 90 | mg |
| | P | 430 | mg |
| | Cl | 350 | mg |
| | Zn | 150 | mg |
| | Retinol (vitamin A) | 0.3 | mg |
| | Calciferol (vitamin D) | 5.0 | mcg |
| | Tocopherol (vitamin E) | 3.0 | mg |
| | Phylloquinone (vitamin K1) | 30.0 | mcg |
| | Thiamin (vitamin B1) | 0.4 | mg |
| | Riboflavin (vitamin B1) | 0.5 | mg |
| | Pyridoxine (vitamin B6) | 0.8 | mg |
| | Cyanocobalamin (vitamin B12) | 0.8 | mcg |
| | Ascorbic acid (vitamin C) | 20.0 | mg |
| | Biotin | 50.0 | mcg |
| | Folic acid | 120.0 | mcg |
| | Niacinamide | 5.0 | mg |
| | Panthothenic acid | 2.0 | mg |
| Energy value | | 229 | kcal |

¹⁾Fraction A1-4C of Example 1 comprising γ-L-glutamyl-trans-S-1-propenyl-L-cysteine sulfoxide.

The above supplement may be mixed with water and taken in appropriate concentration between meals, e.g. between 2 to 4 times daily.

1. Use of γ-glutamyl-peptide in the preparation of a medicament or nutritional formulation for humans or animals for the treatment, testing for or prophylaxis of a disease or condition which is characterized by increased bone resorption.

2. Method of administering to a human or animal who can benefit from an effective amount of γ-glutamyl-peptide.

3. The method as claimed in claim 2 wherein the human or animal is in need of γ-glutamyl-peptide.

4. The method as claimed in claim 2 wherein bone resorption is inhibited.

5. Method of treating, testing for or preventing a disease or condition which is characterized by increased bone resorption comprising administering to a human or animal in need thereof an effective amount of γ-glutamyl-peptide.

6. Use of γ-glutamyl-peptide in the dietary management of increased bone resorption.

7. The method of claim 6 wherein the γ-glutamyl-petide is γ-glutamyl-alkyl-cysteine sulfoxide or γ-glutamy-alkenyl-cysteine sulfoxide, or any combination thereof.

8. The use of claim 1 wherein the γ-glutamyl-alkenyl-cysteine sulfoxide is γ-L-glutamyl-trans-S-1-propenyl-L-cysteine sulfoxide.

9. The use claim 1, wherein the disease or condition which is characterized by increased bone resorption, is Paget's disease, tumor-induced bone disease or osteoporosis or any combination thereof.

10. A nutritional composition comprising γ-glutamyl-peptide and a nutritionally acceptable carrier.

11. The nutritional composition of claim 10 wherein the γ-glutamyl-petide is γ-glutamyl-alkyl-cysteine sulfoxide or γ-glutamy-alkenyl-cysteine sulfoxide or a combination thereof.

12. The nutritional composition of claim 11 wherein the γ-glutamyl-alkenyl-cysteine sulfoxide is γ-L-glutamyl-trans-S-1-propenyl-L-cysteine sulfoxide.

13. The nutritional composition of claim 10 further comprising
(a) a calcium source,
(b) at least one energy source selected from the group consisting of carbohydrate, fat and nitrogen sources, and optionally
(c) Vitamin D.

14. The nutritional composition of claim 13, wherein the calcium source (a) is an organic calcium salt.

15. The nutritional composition of claim 13, wherein the carbohydrate source of component (b) is selected from the group consisting of maltodextrins, starch, lactose, glucose, sucrose, fructose, xylit, sorbit, and mixtures thereof.

16. The nutritional composition of claim 13, wherein the fat source of component (b) is selected from the group consisting of omega-6 polyunsaturated fatty acid sources, omega-3 polyunsaturated fatty acid sources, mono-unsaturated fatty acid sources, C₆-C₁₂-fatty acid sources, and mixtures thereof.

17. The nutritional composition of claim 13, wherein the nitrogen source of component (b) is selected from the group consisting of soy bean derived proteins; milk proteins, protein hydrolysates, a mixture of essential amino acids and arginine, and mixtures thereof.

18. The nutritional composition of claim 13, wherein the carbohydrate source provides for 30 to 70%, the nitrogen source for 5 to 40%, and the fat source for 0.01 to 5% of the total energy supply of the composition.

19. The nutritional composition of claim 13 comprising from 3 to 25% by weight of component (a), from 5 to 50% by weight of component (b) and from 1 to 95% by weight of component (c), based on the total weight of the nutritional composition.

20. The nutritional composition of claim 10 further comprising 0.2 to 10% by weight of other nutritionally acceptable components chosen from vitamins, minerals, trace elements, fibers, flavors, preservatives, colorants, sweeteners and emulsifiers.

21. The nutritional composition of claim 10 in the form of a dietary supplement providing from 50 to 1500 kcal/day, or in the form of an animal feed supplement.

22. The nutritional composition of claim 10 in liquid form.

23. The nutritional composition of claim 10 in granulate or powder form.

24. A pharmaceutical composition in single unit dose form, comprising γ-glutamyl-peptide and a pharmaceutically acceptable carrier.

25. The pharmaceutical composition of claim 24 wherein the γ-glutamyl-petide is γ-glutamyl-alkyl-cysteine sulfoxide or γ-glutamy-alkenyl-cysteine sulfoxide or a combination thereof.

26. The pharmaceutical composition of claim 25 wherein the γ-glutamyl-alkenyl-cysteine sulfoxide is γ-L-glutamyl-trans-S-1-propenyl-L-cysteine sulfoxide.

27. The pharmaceutical composition of claim 24 for enteral administration in the form of a dragée, tablet, capsule, sachet or suppository.

28. The pharmaceutical composition of claim 24 in the form of a veterinary composition.

29. γ-L-glutamyl-trans-S-1-propenyl-L-cysteine sulfoxide obtained by fractionation of an hydrophilic, ethanolic extract of Allium, which fractionation comprises
(a) obtaining an hydrophilic, ethanolic extract of Allium cepa, hereinafter referred to as fraction A, by using adsorption column chromatography,
(b) separating saccharides from fraction A by using reversed-phase medium pressure liquid chromatography (RP-MPLC) to obtain fraction A1
(c) further separating saccharides from fraction A1 by NP-MPLC using chloroform-methanol-water 6.4:5:1 as mobile phase, to obtain fraction A1-4,
(d) further fractionation by semi-preparative reversed-phase HPLC (SP-RP-HPLC) using as solvent an isocratic water/acetonitrile system buffered with e.g. 0.00625% formic acid to obtain fraction A1-4C.

30. The γ-L-glutamyl-trans-S-1-propenyl-L-cysteine sulfoxide of claim 29 wherein said Allium comprises Allium cepa, Allium ascalonicum, Allium ampeloprasum, Allium porrum, Allium schoenoprasum, Allium ursinum, Allium sativum or Allium fistulosum.

31. The γ-L-glutamyl-trans-S-1-propenyl-L-cysteine sulfoxide of claim 30 wherein said allium comprises Allium ascalonicum, Allium porrum, Allium cepa Allium ursinum.

32. The γ-L-glutamyl-trans-S-1-propenyl-L-cysteine sulfoxide of claim 31 wherein said allium comprises allium cepa.

33. Process for producing a veterinary composition for the treatment or prophylaxis of a disease or condition in animal which is characterized by increased bone resorption or for the management of increased bone resorption in animal comprising homogenizing a mixture of one or more carriers that are physiologically acceptable to animals and an effective amount of a γ-glutamyl-peptide.

34. The process of claim 30 wherein the γ-glutamyl-petide is γ-glutamyl-alkyl-cysteine sulfoxide or γ-glutamy-alkenyl-cysteine sulfoxide or a combination thereof.

35. The process of claim 34 wherein the γ-glutamyl-alkenyl-cysteine sulfoxide is γ-L-glutamyl-trans-S-1-propenyl-L-cysteine sulfoxide.

36. The use as claimed in claim 1 wherein γ-glutamyl-peptide inhibits dose-dependently the resorption activity of osteoclasts

37. The use as claimed in claim 1 wherein the minimal effective dose is about 2 mM.

38. The nutritional composition as claimed in claim 10 wherein γ-glutamyl-peptide inhibits dose-dependently the resorption activity of osteoclasts

39. The nutritional composition as claimed in claim 10 wherein the minimal effective dose is about 2 mM.

40. The nutritional composition as claimed in claim 10 wherein the dose is at least 2 mM.

41. The use of claim 1 wherein the γ-glutamyl-petide is γ-glutamyl-alkyl-cysteine sulfoxide or Y-glutamy-alkenyl-cysteine sulfoxide, or any combination thereof.

42. The method of claim 2 wherein the γ-glutamyl-petide is γ-glutamyl-alkyl-cysteine sulfoxide or γ-glutamy-alkenyl-cysteine sulfoxide, or any combination thereof.

43. The method of claim 2 wherein the γ-glutamyl-alkenyl-cysteine sulfoxide is γ-L-glutamyl-trans-S-1-propenyl-L-cysteine sulfoxide.

44. The method of claim 5 wherein the disease or condition which is characterized by increased bone resorption, is Paget's disease, tumor-induced bone disease or osteoporosis or any combination thereof.

45. The nutritional or pharmaceutical composition as claimed in claim 24 wherein γ-glutamyl-peptide inhibits dose-dependently the resorption activity of osteoclasts

45. The nutritional or pharmaceutical composition as claimed in claim 24 wherein the minimal effective dose is about 2 mM.

47. The nutritional or pharmaceutical composition as claimed in claim 24 wherein the dose is at least 2 mM.
